(19) United States
(12) Patent Application Publication
(10) Pub. No.: US 2014/0180043 A1
(43) Pub. Date: Jun. 26, 2014

(54) METHODS AND SYSTEMS FOR DETERMINING SIGNAL QUALITY OF A PHYSIOLOGICAL SIGNAL

(71) Applicant: COVIDIEN LP, Mansfield, MA (US)

(72) Inventors: Paul Stanley Addison, Edinburgh (GB); James Nicholas Watson, Dunfermline (GB)

(73) Assignee: Covidien LP, Mansfield, MA (US)

(21) Appl. No.: 13/726,071

(22) Filed: Dec. 22, 2012

(51) Int. Cl. A61B 5/1455

(52) U.S. Cl. CPC A61B 5/14551
USPC 600/324

(57) ABSTRACT

A physiological monitoring system may use photonic signals at one or more wavelengths to determine physiological parameters. The system may monitor a photoplethysmograph (PPG) signal, which may include a periodic component, and an aperiodic component. An attractor may be generated based on a first segment of the PPG signal and a second segment of the PPG signal shifted in time relative to the first segment by a time delay. The system may analyze points of the attractor that correspond to a curve, analyze the distribution of the attractor about a curve, or both, to determine a signal quality metric indicative of cycle to cycle variation in the PPG signal.

The present disclosure relates to determining signal quality, and more particularly relates to determining signal quality using an attractor.

### SUMMARY

Methods and systems are provided for determining signal quality of a physiological signal.

In some embodiments, a method for determining signal quality of a physiological signal may include receiving a photoplethysmograph signal. The method may include specifying a first segment of the photoplethysmograph signal, where the first segment comprises a first plurality of sample values, and specifying a second segment of the photoplethysmograph signal based on a time delay relative to the first segment, where the second segment comprises a second plurality of sample values. The method may include associating each sample value of the first segment with a corresponding sample value of the second segment to generate a plurality of associated value pairs, and identifying a subset of associated value pairs of the plurality of associated value pairs. When the plurality of associated value pairs are considered in two-dimensional space, the subset of associated value pairs may substantially correspond to a curve. The method may also include determining a signal quality metric based on the subset of associated value pairs. A system may include processing equipment configured to perform the aforementioned method.

In some embodiments, a method for determining signal quality of a physiological signal may include receiving a photoplethysmograph signal. The method may include specifying a first segment of the photoplethysmograph signal, where the first segment comprises a first plurality of sample values, and specifying a second segment of the photoplethysmograph signal based on a time delay relative to the first segment, where the second segment comprises a second plurality of sample values. The method may include associating each sample value of the first segment with a corresponding sample value of the second segment to generate a plurality of associated value pairs, and determining one or more metrics. The one or more metrics may be indicative of how the plurality of associated value pairs, when considered in two-dimensional space, are distributed about a curve. The method may also include determining a signal quality metric based on the one or more metrics. A system may include processing equipment configured to perform the aforementioned method.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system, in accordance with some embodiments of the present disclosure;

FIG. 2 is a block diagram of the illustrative patient monitoring system of FIG. 1 coupled to a patient, in accordance with some embodiments of the present disclosure;

FIG. 3 shows a block diagram of an illustrative signal processing system in accordance with some embodiments of the present disclosure;

FIG. 4 shows an illustrative signal that may be analyzed in accordance with some embodiments of the present disclosure;

FIG. 5 shows a plot of an illustrative periodic signal, in accordance with some embodiments of the present disclosure;

FIG. 6 shows six plots of illustrative value pairs based on the periodic signal of FIG. 5 for different time delays, in accordance with some embodiments of the present disclosure;

FIG. 7 show a plot of an illustrative photoplethysmograph signal, in accordance with some embodiments of the present disclosure;

FIG. 8 shows a plot of an illustrative attractor generated from the photoplethysmograph signal of FIG. 7, in accordance with some embodiments of the present disclosure;

FIG. 9 is a flow diagram of illustrative steps for determining signal quality based on a subset of associated value pairs, in accordance with some embodiments of the present disclosure;

FIG. 10 is a flow diagram of illustrative steps for determining signal quality based on associated value pairs, in accordance with some embodiments of the present disclosure;

FIG. 11 shows a plot of the illustrative attractor of FIG. 8 and a reference curve, in accordance with some embodiments of the present disclosure;

FIG. 12 shows a histogram of intersections of the illustrative attractor and reference curve of FIG. 11, in accordance with some embodiments of the present disclosure;

FIG. 13 shows a plot of an illustrative PPG signal, in accordance with some embodiments of the present disclosure;

FIG. 14 shows a plot of associated value pairs for a time delay substantially equal to a period of a physiological rate, in accordance with some embodiments of the present disclosure;

FIG. 15 shows a plot of an illustrative attractor in three-dimensions generated using associated value triples, and a reference surface, in accordance with some embodiments of the present disclosure; and

FIG. 16 shows intersections of the attractor and the reference surface of FIG. 15, viewed normal to the reference surface, in accordance with some embodiments of the present disclosure.

### DETAILED DESCRIPTION OF THE FIGURES

The present disclosure is directed towards determining a signal quality metric based on an attractor generated from segments of a photoplethysmograph signal. The PPG signal may be generated using, for example, an oximeter such as a pulse oximeter.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I₀(λ)exp(−(sβ₀(λ)+(1−s)βᵣ(λ))l(t)) (1)

where:
λ=wavelength;
t=time;
I=intensity of light detected;
I₀=intensity of light transmitted;
s=oxygen saturation;
β₀, βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I₀−(sβ₀+(1−s)βᵣ)l. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λₙ, in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{dI / dt}{I} , \qquad ( 10 )$$

Eq. 8 becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{\frac{I ( {t}_{2} , {λ}_{IR} ) - I ( {t}_{1} , {λ}_{IR} )}{I ( {t}_{1} , {λ}_{IR} )}}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

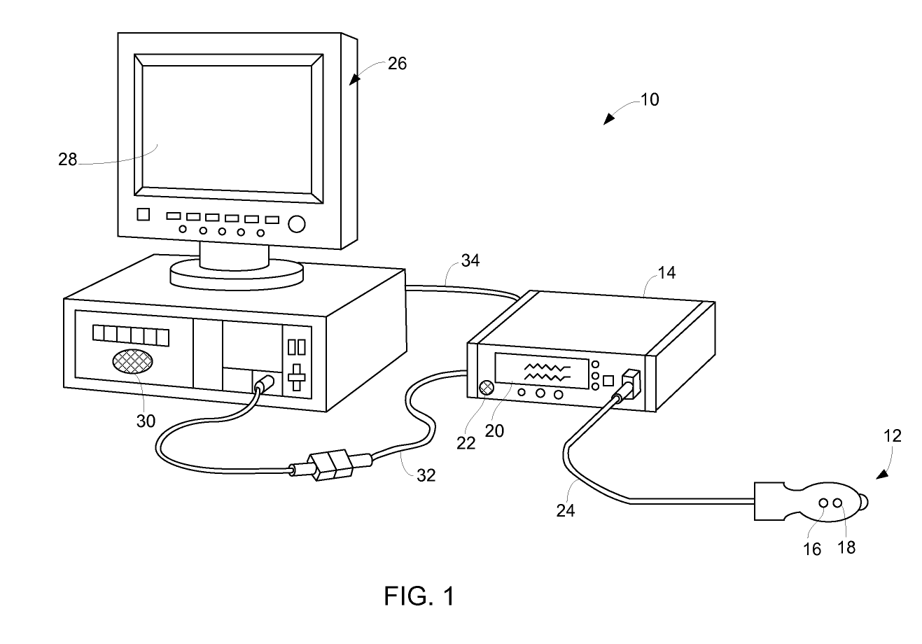

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to another embodiment, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood pressure, blood oxygen saturation) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the monitor 14 includes a blood pressure monitor. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may be allow communication between monitor 14 and sensor unit 12.

In the illustrated embodiment, system 10 includes a multi-parameter patient monitor 26. The monitor 26 may include a cathode ray tube display, a flat panel display (as shown) such as a liquid crystal display (LCD) or a plasma display, or may include any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by monitor 14 (referred to as a “SpO₂” measurement), pulse rate information from monitor 14 and blood pressure from monitor 14 on display 28. Multi-parameter patient monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

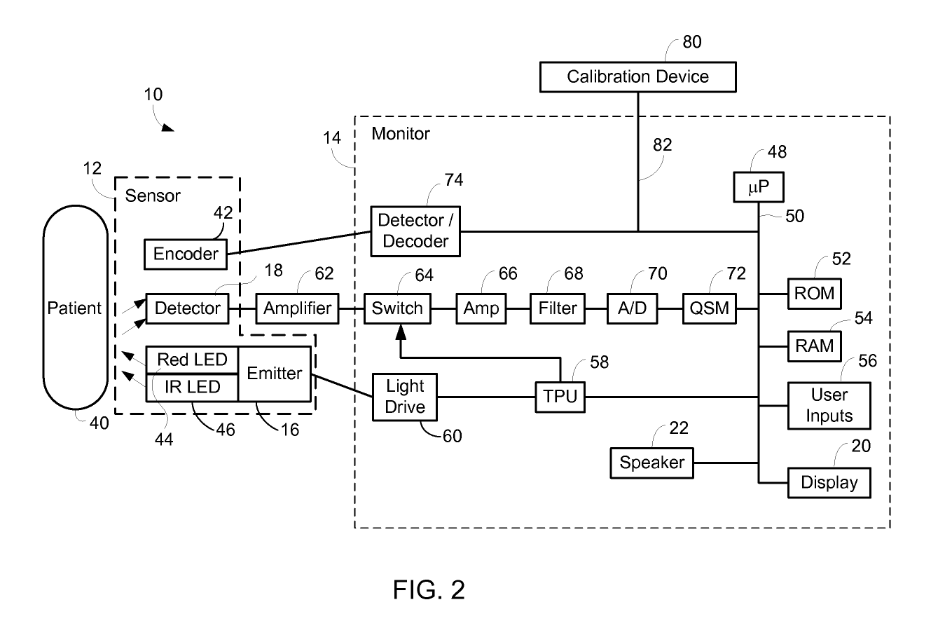

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor emits only a Red light while a second emits only an IR light. In a further example, the wavelengths of light used are selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a portion of a photoplethysmograph (PPG) signal corresponding to a physiological pulse to determine blood pressure. These equations may contain coefficients that depend upon a patient's physiological characteristics as stored in encoder 42. Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics. In some embodiments, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment.”

In some embodiments, microprocessor 48 may determine the patient's physiological parameters, such as SpO₂, pulse rate, and/or blood pressure, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. In some embodiments, user inputs 56 may be used enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

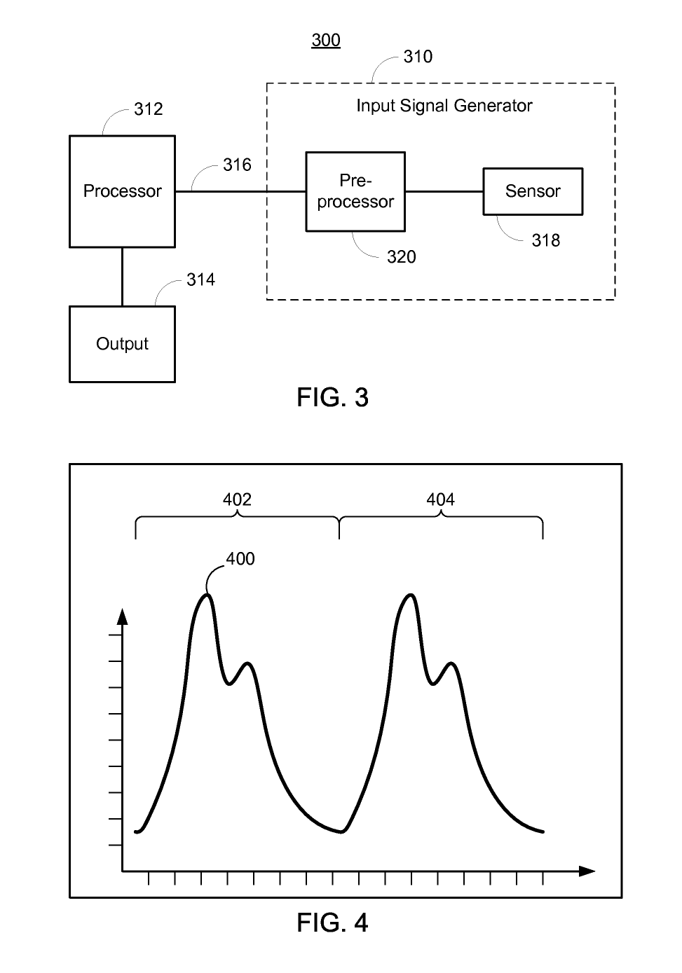

FIG. 3 is an illustrative signal processing system 300 in accordance with an embodiment that may implement the signal processing techniques described herein. In some embodiments, signal processing system 300 may be included in a patient monitoring system (e.g., patient monitoring system 10 of FIGS. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be an oximeter and input signal 316 may be a PPG signal. In an embodiment, pre-processor 320 may be any suitable signal processing device and input signal 316 may include one or more PPG signals and one or more other physiological signals, such as an electrocardiogram (ECG) signal. It will be understood that input signal generator 310 may include any suitable signal source, signal generating data, signal generating equipment, or any combination thereof to produce signal 316. Signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, signal 316 may include PPG signals corresponding to one or more light frequencies, such as a Red PPG signal and an IR PPG signal. In some embodiments, signal 316 may include signals measured at one or more sites on a patient's body, for example, a patient's finger, toe, ear, arm, or any other body site. In some embodiments, signal 316 may include multiple types of signals (e.g., one or more of an ECG signal, an EEG signal, an acoustic signal, an optical signal, a signal representing a blood pressure, and a signal representing a heart rate). Signal 316 may be any suitable biosignal or signals, such as, for example, electrocardiogram, electroencephalogram, electrogastrogram, electromyogram, heart rate signals, pathological sounds, ultrasound, or any other suitable biosignal. The systems and techniques described herein are also applicable to any dynamic signals, non-destructive testing signals, condition monitoring signals, fluid signals, geophysical signals, astronomical signals, electrical signals, financial signals including financial indices, sound and speech signals, chemical signals, meteorological signals including climate signals, any other suitable signal, and/or any combination thereof.

In some embodiments, signal 316 may be coupled to processor 312. Processor 312 may be any suitable software, firmware, hardware, or combination thereof for processing signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may locate one or more reference points on one or more signals, which may be found by performing mathematical calculations on the signal. In a further example, processor 312 may locate one or more fiducial points on one or more signals, and compute one or more of a pulse rate, respiration rate, blood pressure, or any other suitable physiological parameter. Processor 312 may perform any suitable signal processing of signal 316 to filter signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the patient. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 312 to, for example, store fiducial information corresponding to physiological monitoring. In some embodiments, processor 312 may store physiological measurements or previously received data from signal 316 in a memory device for later retrieval. In some embodiments, processor 312 may store calculated values, such as a blood pressure, a blood oxygen saturation, a pulse rate, a fiducial point location or characteristic, or any other calculated values, in a memory device for later retrieval.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as part of sensor unit 12 (FIGS. 1 and 2) and monitor 14 (FIGS. 1 and 2) and processor 312 may be implemented as part of monitor 14 (FIGS. 1 and 2). In some embodiments, portions of system 300 may be configured to be portable. For example, all or part of system 300 may be embedded in a small, compact object carried with or attached to the patient (e.g., a watch, other piece of jewelry, or a smart phone). In some embodiments, a wireless transceiver (not shown) may also be included in system 300 to enable wireless communication with other components of system 10 (FIGS. 1 and 2). As such, system 10 (FIGS. 1 and 2) may be part of a fully portable and continuous patient monitoring solution. In some embodiments, a wireless transceiver (not shown) may also be included in system 300 to enable wireless communication with other components of system 10. For example, pre-processor 320 may output signal 316 over BLUETOOTH, 802.11, WiFi, WiMax, cable, satellite, Infrared, or any other suitable transmission scheme. In some embodiments, a wireless transmission scheme may be used between any communicating components of system 300.

Pre-processor 320 or processor 312 may determine the locations of pulses within a periodic signal 316 (e.g., a PPG signal) using a pulse detection technique. For ease of illustration, the following pulse detection techniques will be described as performed by processor 312, but any suitable processing device (e.g., pre-processor 320) may be used to implement any of the techniques described herein.

An illustrative PPG signal 400 is depicted in FIG. 4. For example, processor 312 may receive PPG signal 400 from sensor 318. PPG signal 400 includes two periods 402 and 404 (e.g., corresponding to a pulse rate of a subject) of physiological data, although a PPG signal may include continuous data of multiple periods. Each period of physiological data, including an upstroke and a downstroke (and any notches that may be present) is referred to as a pulse wave. PPG signal 400 may have some substantial periodic character, as well as aperiodic components such as baseline shifts, amplitude shifts, phase shifts, noise, or other components. In some embodiments, the techniques described herein may provide a quantification and/or qualification of the periodic character of physiological data.

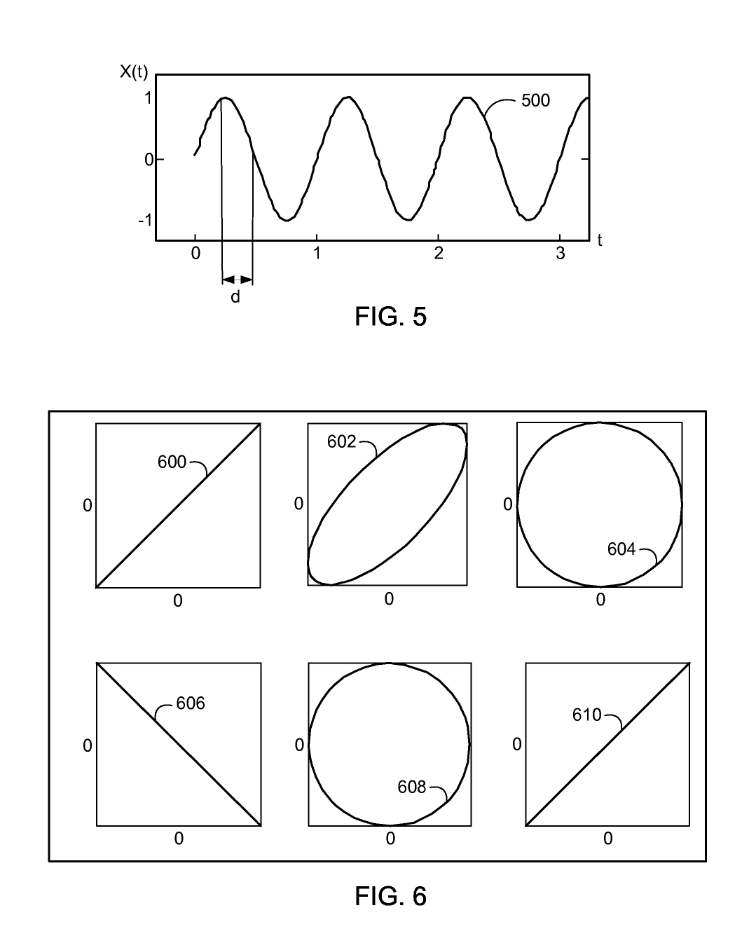

FIG. 5 show a plot of an illustrative periodic signal 500, in accordance with some embodiments of the present disclosure. Periodic signal 500, a sine wave having a period and amplitude of one, is a relatively simple periodic signal for purposes of illustration. In some embodiments, a set of value pairs may be generated by pairing values of periodic signal 500, for example f(t) at discrete time t values, with values of period signal 500 delayed in time, for example f(t+d) at the same discrete time t values, where d is the time delay, as shown in FIG. 5. The set of value pairs is referred to herein as an attractor. FIG. 6 shows six plots of illustrative value pairs based on the periodic signal 500 of FIG. 5 for different time delays d, in accordance with some embodiments of the present disclosure. The six attractors 600, 602, 604, 606, 608, and 610 correspond to time delays of 0, ⅛ period, ¼ period, ½ period, ¾ period, and 1 period, respectively. Note that the attractors correspond to Lissajous curves in this example. Referencing attractor 600, for a time delay of 0, the value pairs are coincident with the line “y=x.” As the time delay is increased from 0 to an eighth period, the value pairs tend to attractor 602. As the time delay is further increased to a quarter period, the value pairs tend to attractor 604, which forms a circle. As the time delay is increased from a quarter period to half period, the value pairs tend to attractor 606, for which the value pairs are coincident with the line “y=−x.” As the time delay is further increased to three quarter period, the value pairs tend to attractor 608, which forms a circle. Finally, for a time delay of one period, the value pairs are again coincident with the line “y=x.” It can be seen from FIG. 6 that attractors open up to a maximum at one-quarter period and three-quarter period, where a circular attractor is generate, and that the attractors close up when the delay is zero and when it is one half cycle, one cycle, and integer multiples thereof. When a repeating signal of a more complex geometry is analyzed, the corresponding attractors may only close up when the time delay is an integer multiple of the signal period. The attractor will only close up if the repeating waveform is exactly the same during each cycle (e.g., there are no fluctuations in the signal longer than the cycle period under investigation). It will be understood that aperiodicity, and more complex waves result in more complex attractors that may or may not form closed curves or lie on a line. For example, referencing a time delay of one period, the value pairs may be scattered about the line “y=x” due to changes from one pulse wave to the next. In a further example, referencing a time delay of a quarter period or three-quarter period, the value pairs may or may not correspond to circles. In some embodiments, a signal may be centered about zero (e.g., by performing a mean subtraction), so that the corresponding attractor is substantially centered about the origin. If a signal is not centered about zero, corresponding attractors may be substantially centered about points other than the origin. Details regarding attractors, and analysis thereof, may be found in the book “Fractals and Chaos: An illustrated Course” by Paul S. Addison, 1997, which is hereby incorporated by reference herein in its entirety.

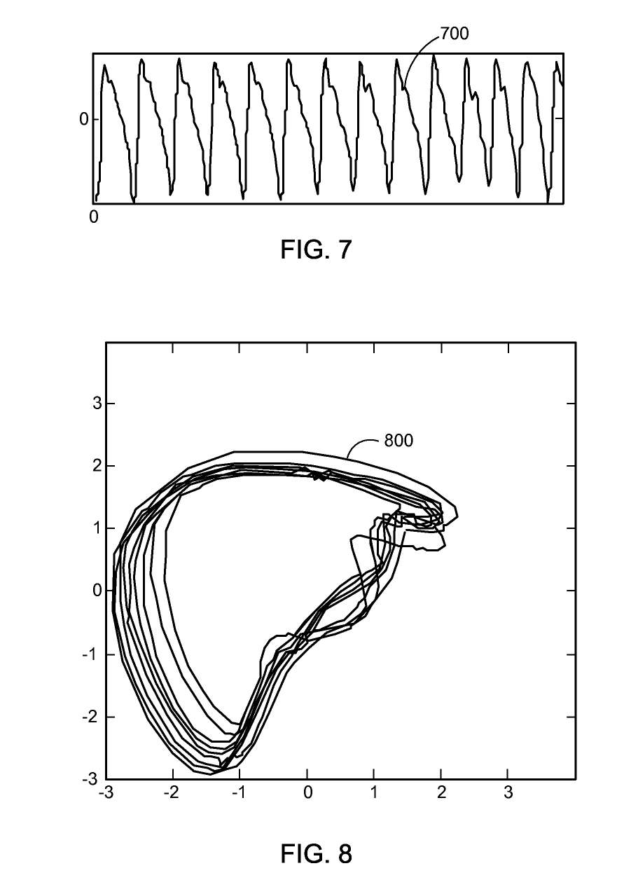

In some embodiments, an attractor need not be a closed, relatively simple curve. FIG. 7 shows a plot of an illustrative PPG signal 700, in accordance with some embodiments of the present disclosure. PPG signal 700 includes multiple pulse waves, centered about zero. FIG. 8 show a plot of an illustrative attractor 800 generated from PPG signal 700 of FIG. 7, using a time delay of approximately a quarter period, in accordance with some embodiments of the present disclosure. Attractor 800 is not a circle, due to the shape of pulse waves of PPG signal 700. Also, attractor 800 is not a closed curve (nor do the value pairs lie on a line), but rather includes a sequence of nearly closed shapes (each corresponding to one period, referred to as a cycle), which are substantially similar but not identical, having some variation due to variability in PPG signal 700. The variability in the sequence of shapes provides an indication of the variability of the signal. In some embodiments, an attractor may be analyzed to determine the stability (e.g., similarity, or lack of variation, from cycle to cycle) of the attractor. It will be understood that processing equipment need not plot an attractor to analyze the attractor, and FIGS. 5-8 are provided as graphical examples.

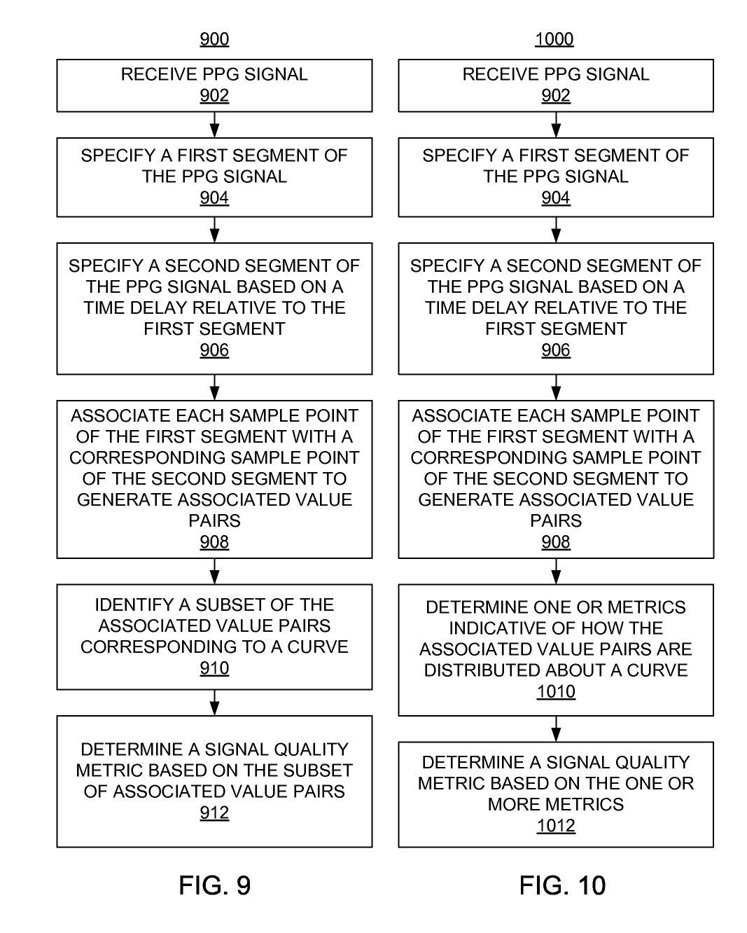

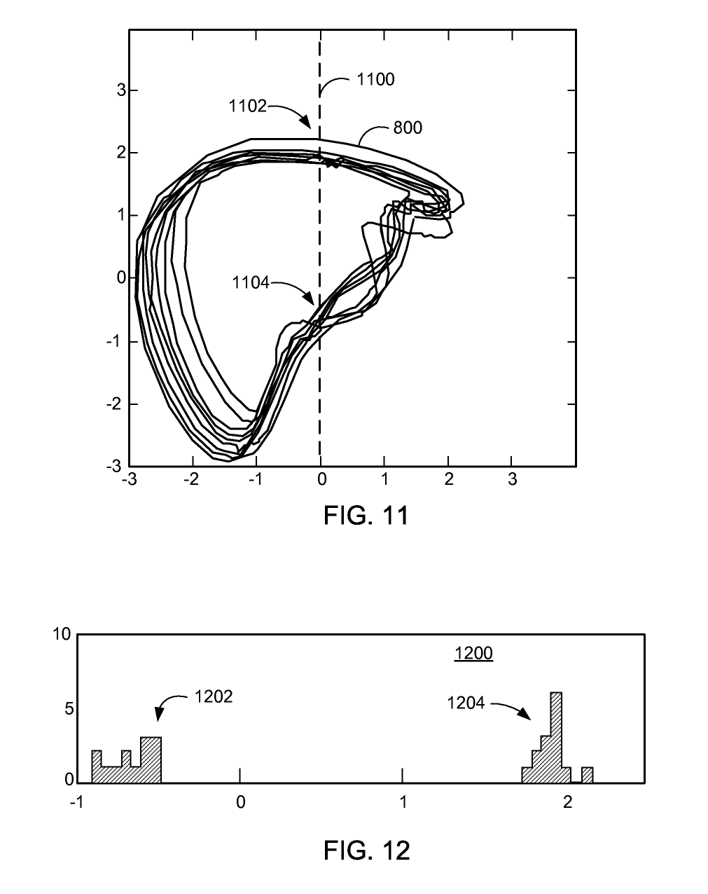

FIGS. 9 and 10 are flow diagrams 900 and 1000 of illustrative steps for determining signal quality. FIGS. 11-16, which provide graphical examples of some techniques of the present disclosure, will be referenced in the context of flow diagrams 900 and 1000.

FIG. 9 is a flow diagram 900 of illustrative steps for determining signal quality based on a subset of associated value pairs, in accordance with some embodiments of the present disclosure.

Step 902 may include processing equipment receiving a PPG signal from a physiological sensor, memory, any other suitable source, or any combination thereof. For example, referring to system 300 of FIG. 3, the processing equipment may receive a window of physiological data from input signal generator 310. Sensor 318 of input signal generator 310 may be coupled to a subject, and may detect physiological activity such as, for example, RED and/or IR light attenuation by tissue, using a photodetector. In some embodiments, physiological signals generated by input signal generator 310 may be stored in memory (e.g., RAM 54 of FIG. 2, QSM 72 and/or other suitable memory) after being pre-processed by pre-processor 320. In such cases, step 902 may include recalling data from the memory for further processing. In some embodiments, the processing equipment may filter the PPG signal. For example, the processing equipment may apply a high-pass filter (e.g., having a cutoff frequency below the expected heart rate) to reduce or substantially remove baseline changes and other low-frequency artifacts. In a further example, the processing equipment may apply a low-pass filter (e.g., having a cutoff frequency above the expected heart rate) to reduce or substantially remove higher frequency noise or features. In a further example, the processing equipment may apply a bandpass filter to reduce or substantially remove low and high frequency artifacts and features. In some embodiments, steps 904-912 may be based on a Red PPG signal, IR PPG signal, a derivative thereof, a processed signal derived thereof, or any combination thereof.

Step 904 may include the processing equipment specifying a first segment of the PPG signal of step 902. In some embodiments, the first segment of the PPG signal may include a particular number of sample points. In some embodiments, selecting the first segment of the PPG signal may include specifying indices of sample points in the PPG signal. The length of the first segment, in time or sample number, may be any suitable length. For example, the length may be equal to a period corresponding to a physiological rate (e.g., a heart rate), or multiple thereof.

Step 906 may include the processing equipment specifying a second segment of the PPG signal of step 902 based on a time delay relative to the first segment of step 904. The second segment includes the same number of samples points as the first segment, albeit shifted in time by a time delay. Accordingly, for time delays of less than the size of the first segment, the first and second segments may share one or more sample points, although the shared points may be located relatively differently in the respective segments. In some embodiments, selecting the second segment of the PPG signal may include specifying indices of sample points in the PPG signal.

In some embodiments, the time delay may be predetermined. In some embodiments, the time delay may be determined based on an analysis of the resulting attractor. For example, the time delay may be predetermined to be a quarter period (e.g., where the period correspond to a physiological rate), or any other suitable fraction or multiple of the period. In a further example, the time delay may be determined to result in a maximum variation in the attractor from cycle to cycle (e.g., based on correspondence to a curve). In a further example, the time delay may be determined based on a minimum mutual information analysis of the first and second segments. Any suitable technique may be used to determine or select a time delay. In some embodiments, the time delay may be determined based on morphology of the PPG signal. In some embodiments, the time delay may be determined based on a population-based parametric study of PPG signals. In some embodiments, the time delay may be determined from an autocorrelation of the signal. For example, the time delay may be determined as a fraction (e.g., one quarter) of the time between two consecutive peaks in the autocorrelation.

Step 908 may include the processing equipment associating each sample point of the first segment with a corresponding sample point of the second segment to generate associated value pairs. The associated value pairs include a first value and a second value from the PPG signal of step 902, spaced in time or sample number by the time delay. In some embodiments, the associated value pairs are generated by associating sample points of the first and second segments by relative indices within the respective segments. For example, the first point of the first segment may be associated with the first point of the second segment, the second point of the first segment may be associated with the second point of the second segment, and so on. In some embodiments, the processing equipment may store the associated value pairs, indices corresponding to the associated value pairs, or both.

Step 910 may include the processing equipment identifying a subset of the associated value pairs corresponding to a curve, when the associated value pairs are considered in two-dimensional space. In some embodiments, the curve may be a line, polynomial of 2 or higher, a piecewise curve, any other suitable curve, or any combination thereof. For example, when considered in two-dimensional space, the curve may be a horizontal line, a vertical line, an oblique line, or piecewise combination thereof. In some embodiments, identifying the subset of the associated value pairs corresponding to the curve may include identifying intersections of the associated value pairs and the curve. For example, the associated value pairs nearest the curve may be identified. In a further example, adjacent associated value pairs on either side of the curve may be identified, and an interpolated associated value pair coincident with the curve may be determined. In a further example, multiple associated value pairs may be identified (e.g., each corresponding to one period's worth of the first segment) and a histogram may be generated based on the multiple pairs.

In some embodiments, step 910 may include selecting, or otherwise generating, the curve. In some embodiments, the curve used may depend on the time delay. For example, because the attractor is expected to close as the time delay is nearer to a half period or full period (or multiple thereof), the curve used for such time delays may differ from a curve used for time delays nearer to a quarter of a period or three-quarter period. In some embodiments, the curve may depend on a physiological rate. For example, different curves may be used for small heart rates relative to large heart rates. In some embodiments, the curve can be selected to be perpendicular to the attractor. In some embodiments, the curve can be applied to an optimal location in the attractor (e.g., where the cycle to cycle spread of the attractor is a minimum or likely to be a minimum).

Step 912 may include the processing equipment determining a signal quality metric based on the subset of associated value pairs identified at step 910. The signal quality metric may be indicative of variability, or the lack of variability thereof, in the PPG signal from cycle to cycle. In some embodiments, the processing equipment may determine the signal quality metric based on collective calculations performed on the subset of associated value pairs.

In an illustrative example of the techniques of flow diagram 900, FIG. 11 show a plot 1100 of illustrative attractor 800 of FIG. 8 and a reference curve 1100, in accordance with some embodiments of the present disclosure. Reference curve 1100 is a vertical line (e.g., given by equation x=0). Vertical and horizontal lines may provide convenient curves, because the identification of intersections (e.g., intercepts) is relatively easy. For example, referencing FIG. 11, the intersections of the attractor (i.e., associated value pairs 800) denoted by arrows 1102 and 1104 are the “y-intercepts” and may be identified by finding zeros or near zeros in the first value of each associated value pair. However it will be understood that any suitable curve, linear or otherwise, of any suitable orientation, may be used. In some embodiments, more than one curve may be used. For example, a second vertical line may be added to FIG. 11 (not shown), and a further subset of associated value pairs corresponding to the second line may be identified. Any suitable number of curves may be used in accordance with the present disclosure. FIG. 12 show a histogram 1200 of intersections of attractor 800 and reference curve 1100 of FIG. 11, in accordance with some embodiments of the present disclosure. The abscissa of FIG. 12 is in units along curve 1100, while the ordinate of FIG. 12 is the number of intersections. PPG signals having relatively low variation from pulse wave to pulse wave are expected to exhibit a tighter histogram (e.g., a higher peak and smaller spread), while PPG signals having greater variability are expected to exhibit a wider, more spread out histogram. Histogram 1200 exhibits two peaks, indicated by arrows 1202 and 1204, which correspond to the two sets of intersections of attractor 800 and reference curve 1100.

In some embodiments, at step 912 the processing equipment may determine a mean location (e.g., center location of a peak), standard deviation (e.g., standardized deviation of the peak from the center), median deviation, entropy, any other suitable metric derived from the subset of associated value pairs, or any combination of metrics thereof, as a measure of signal quality. For example, the processing equipment may determine the mean location of a peak (e.g., peaks indicated by arrow 1202 and/or 1204) and compare it with predetermined thresholds to determine a signal quality metric. In a further example, the processing equipment may determine the standard deviation of a peak of histogram 1200 and compare it with a threshold. If the standard deviation exceeds the threshold, the processing equipment may categorize the PPG signal as having low quality. In a further example, a peak in a generated histogram may be compared with a reference distribution, and a similarity metric may be determined. In a further example, a determined metric itself may be used as a signal quality metric. In a further example, a determined metric may be scaled and used as a signal quality metric. In a further example, a determined metric may be input into a function or look-up table which outputs a signal quality metric.

FIG. 10 is a flow diagram 1000 of illustrative steps for determining signal quality based on associated value pairs, in accordance with some embodiments of the present disclosure. In some embodiments, the illustrative techniques of flow diagram 1000 may include performing steps similar to step 902-908 of flow diagram 900 of FIG. 9 to generate associated value pairs.

Step 1010 may include the processing equipment determining one or more metrics indicative of how the associated value pairs are distributed about a curve. The processing equipment may, for example, determine a variance, standard deviation, median absolute deviation, any other metric indicative of the deviation of the associated value pairs from the curve, or any combination thereof. In some embodiments, the processing equipment may determine the distribution of associated values pairs about the curve (or along the curve), and compare the distribution with a reference distribution.

In some embodiments, the curve used at step 1010 may be selected or generated based on the associated value pairs. For example, the curve may be a best fit line (e.g., using a linear regression technique), or other best fit curve, determined based on the associated value pairs. In some embodiments, the curve may be predetermined. For example, the curve may be a line “y=x,” a unit circle, any other suitable open or closed curve, or any combination thereof. In some embodiments, the curve may be selected from multiple curves based on the time delay. For example, if the time delay is a multiple of the period, the processing equipment may select the curve “y=x.” In a further example, if the time delay is an odd multiple of half the period, the processing equipment may select the curve “y=−x.” In a further example, if the time delay is a quarter period or three-quarter period, the processing equipment may select a circle (e.g., a unit circle if the PPG signal is normalized between 1 and −1 similar to attractor 604 or 608 of FIG. 6) as the curve. In a further example, if the time delay is an eighth period, the processing equipment may select an ellipse (e.g., similar to attractor 602 of FIG. 6) as the curve. Any suitable curve may be used at step 1010 in accordance with the present disclosure. The curve may be open, closed, defined piecewise, continuous, discontinuous, defined by a function, defined by a sequence of point, defined by a relationship other than a function, or any combination thereof. In some embodiments, a best fit curve may be fit to the associated value pairs, and compared with a reference best fit curve.

Step 1012 may include the processing equipment determining a signal quality metric based on the one or more metrics determined at step 1010. The signal quality metric may be indicative of how well the attractor agrees with the curve. The signal quality metric may be equal to one of the one or more metrics, which may be shifted or scaled, derived from a function or look-up table using the one or more metrics as inputs. In some embodiments, the signal quality metric may be outputted for display to a user, for example, in the form of a number, bar, icon, or other visual indicator.

In some embodiments, the quality metric may be used by the system to assist in determining physiological information. For example, data may be disqualified if the signal quality is too low. In a further example, the system may filter or process data depending on the signal quality level. In some embodiments, a signal quality metric can be used to vary the filtering of physiological parameters. For example, for data corresponding to low signal quality, the most recently calculated physiological parameter may be weighted less when averaging it with previously calculated values.

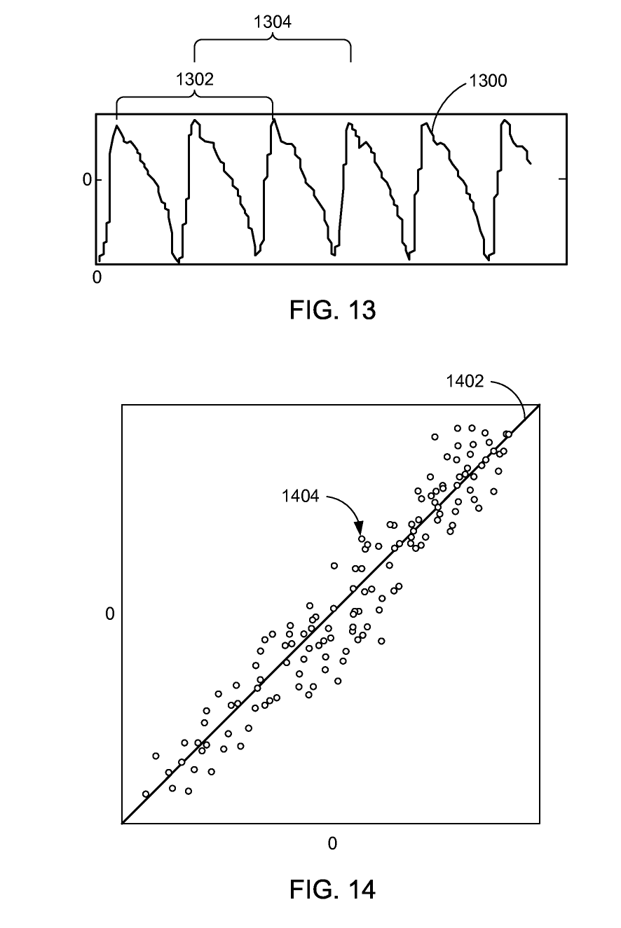

In an illustrative example of the techniques of flow diagram 1000, FIG. 13 show a plot of illustrative PPG signal 1300, in accordance with some embodiments of the present disclosure. First segment 1302 includes about two periods of PPG signal 1300. Second segment 1304 also includes about two periods of PPG signal 1300, at a time delay of about one period from first segment 1302. FIG. 14 show a plot 1400 of illustrative associated value pairs derived from a PPG signal such as PPG signal 1300 of FIG. 13, for a time delay substantially equal to a period of a physiological rate, in accordance with some embodiments of the present disclosure. The processing equipment may, for example, compare associated value pairs 1404 with curve 1402, which is the line “y=x”. The processing equipment may determine a deviation of associated value pairs 1404 from curve 1402 using any suitable technique. For example, the processing equipment may use any of the following Eqs. 14-16 do determine a signal quality metric M:

$$\begin{matrix}M = {\sum }_{i = 1}^{N} {( {y}_{i} - {C}_{i} )}^{2} & ( 14 ) \\ M = {\sum }_{i = 1}^{N} \vert {y}_{i} - {C}_{i} \vert & ( 15 ) \\ M = \sqrt{\frac{1}{N} {\sum }_{i = 1}^{N} {( {y}_{i} - {C}_{i} )}^{2}} & ( 16 )\end{matrix}$$

in which yᵢ is the second value (e.g., vertical value in FIG. 14) of the i^{th} associated value pair of N pairs and Cᵢ is the value of the curve sharing a first value (e.g., horizontal value in FIG. 14) with the i^{th} associated value pair. Eqs. 14-16 are merely illustrative, and any suitable metric, such as the mean absolute deviation and the median absolute deviation, may be determined based on the associated value pairs and the curve. In an illustrative example, the processing equipment may normalize a PPG signal to vary between −1 and 1, use a time delay of a quarter period, and use a unit circle as a reference curve rather than a line. The processing equipment may determine a signal quality metric using any of Eqs. 14-16, or variations thereof, or any other equations, to quantify the distribution of the resulting attractor and the unit circle.

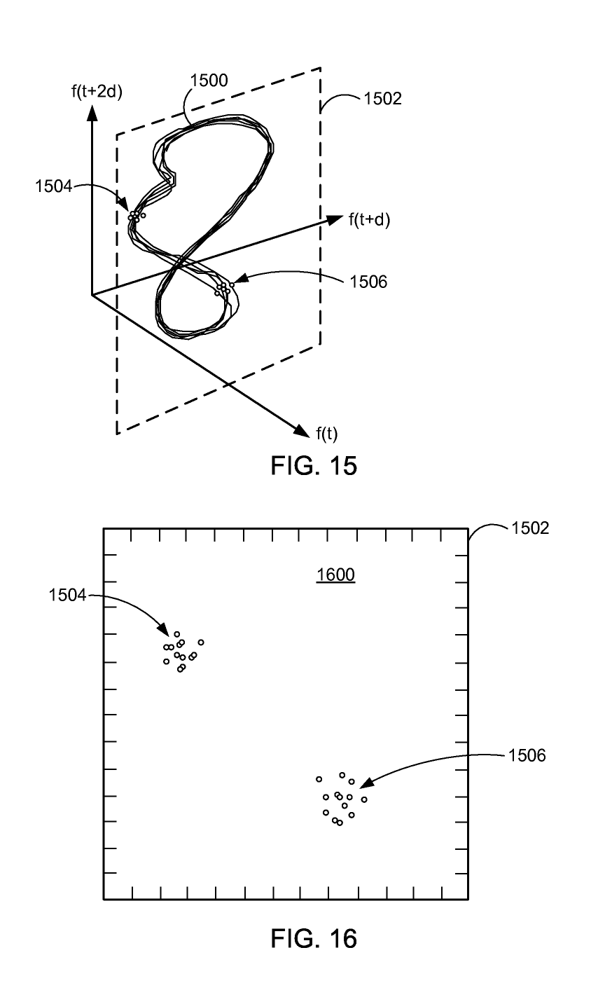

In some embodiments, the processing equipment may generate an attractor in more than two dimensions. For example, the processing equipment may generate associated value groups of more than two (e.g., associated value triples). FIG. 15 show a plot of an illustrative attractor 1500 in three-dimensions generated using associated value triples, and a reference surface 1502, in accordance with some embodiments of the present disclosure. In the illustrated example of FIG. 15, reference surface 1502 is a plane (e.g., which may include a sequence of curves, or a curve generalized to three dimensions). Attractor 1500 includes associated value triples, in which the first value is f(t), the second value is f(t+d), and the third value is f(t+2d), where d is the time delay, and 2d is twice the time delay. The delays d and 2d are illustrative, and any suitable time delays may be used to generate associated value triples. Attractor 1502 intersects reference surface 1504 in two regions, indicated by arrows 1504 an 1506.

FIG. 16 shows intersections of attractor 1500 and the reference surface 1502, viewed normal to reference surface 1502 (i.e., in the −f(t) direction), in accordance with some embodiments of the present disclosure. In the illustrated example, two-dimensional space 1600 is coincident with reference surface 1502. The two groupings of intersections, indicated by open circles in FIG. 16, between attractor 1500 and reference surface 1502 are indicated by arrows 1504 and 1506. Any of the illustrative techniques of flow diagram 900 or 1000 may be applied in dimensions greater than two. For example, the processing equipment may determine a mean location, standard deviation, median variance, entropy, distribution, any other suitable metric or result from which a metric may be derived, or any combination thereof, for each grouping of intersections indicated by arrows 1504 and 1506. For example, for each group of intersections, the processing equipment may determine the root mean square (RMS) distance of the intersections from the mean location and compare the RMS distance to a threshold. In a further example, for each group of intersections, the processing equipment may determine the mean location of the intersections from the mean location and compare the mean location to a reference location. In a further example, for each group of intersections, the processing equipment may determine the entropy of the intersections and compare the entropy to a reference location. In a further example, for each group of intersections, the processing equipment may determine a distribution of intersections about a mean location and compare the distribution to a reference distribution.

In a further illustrative example, one or more attractors may be generated for multiple (e.g., two or more) delays, and the attractor may be analyzed according to the techniques of both flow diagrams 900 and 1000. For example, associated value triples may be generated using f(t), f(t+p/4), and f(t+p), where “p” is the period. The processing equipment may analyze the values corresponding to f(t) and f(t+p/4) in accordance with flow diagram 900, and analyze the values corresponding to f(t) and f(t+p) in accordance with flow diagram 1000. In some embodiments, the processing equipment may project an attractor into any suitable number of dimensions to determine a signal quality metric.

The techniques disclosed herein may be used to interrogate both the Red PPG signal and the IR PPG signal. The system may analyze the RED and IR PPG signals independently, or may analyze the signals in concert. For example, using a time delay substantially equal to the period, both signals may be compared to a 45 degree line (e.g., the line y=x). In a further example, if analyzed independently, the two signal quality metrics may be averaged, the worse signal quality metric may be selected, or the better signal quality metric may be selected, as the final value. In a further example, if analyzed in concert, the Red and IR PPG signals may be averaged, and the averaged signal may be compared to a curve. In a further example, the system may pair the values of the Red and IR PPG signals with no time delay, and the illustrative steps flow diagram 1000 of FIG. 10 may be performed on the value pairs. In a further example, the system may pair the values of the Red and IR PPG signals with a time delay, and the illustrative steps of flow diagram 900 of FIG. 9 may be performed on the value pairs.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for determining signal quality of a physiological signal, the method comprising:
receiving a photoplethysmograph signal;
specifying, using processing equipment, a first segment of the photoplethysmograph signal, wherein the first segment comprises a first plurality of sample values;
specifying, using the processing equipment, a second segment of the photoplethysmograph signal based on a time delay relative to the first segment, wherein the second segment comprises a second plurality of sample values;
associating, using the processing equipment, each sample value of the first segment with a corresponding sample value of the second segment to generate a plurality of associated value pairs;
identifying, using the processing equipment, a subset of associated value pairs of the plurality of associated value pairs, wherein when the plurality of associated value pairs are considered in two-dimensional space, the subset of associated value pairs substantially correspond to a curve; and
determining, using the processing equipment, a signal quality metric based on the subset of associated value pairs.

2. The method of claim 1, wherein the curve comprises a line.

3. The method of claim 1, wherein identifying the subset of associated value pairs of the plurality of associated value pairs comprises:
identifying at least one set of two associated values pairs that, when considered in the two-dimensional space, lie on either side of the curve; and
for each identified set of two associated value pairs, determining a respective intersection value by interpolating a point on the curve between the two associated value pairs, wherein determining the signal quality metric is further based on the at least one intersection value.

4. The method of claim 1, wherein determining a signal quality metric comprises determining a variability metric associated with the subset of associated value pairs, wherein the variability metric is selected from the group comprising an average magnitude, a standard deviation, a median deviation, and an entropy value.

5. The method of claim 1, wherein the first plurality of sample values and the second plurality of sample values comprise the same number of samples values, and wherein a time interval corresponding to the number of sample values is greater than or substantially equal to a characteristic period of the photoplethysmograph signal.

6. The method of claim 1, further comprising:
specifying a third segment of the photoplethysmograph signal based on a second time delay relative to the first segment, wherein the third segment comprises a third plurality of sample values; and
associating each of the plurality of associated value pairs with a correspond sample value of the third segment to generate a plurality of associated value triples;
identifying a subset of associated value triples of the plurality of associated value triples, wherein when the plurality of associated value triples are considered in three-dimensional space, the subset of associated value triples substantially correspond to a surface in the three-dimensional space; and
determining the signal quality metric based on the subset of associated value triples.

7. A method for determining signal quality of a physiological signal, the method comprising:
receiving a photoplethysmograph signal;
specifying, using processing equipment, a first segment of the photoplethysmograph signal, wherein the first segment comprises a first plurality of sample values;
specifying, using the processing equipment, a second segment of the photoplethysmograph signal based on a time delay relative to the first segment, wherein the second segment comprises a second plurality of sample values;
associating, using the processing equipment, each sample value of the first segment with a corresponding sample value of the second segment to generate a plurality of associated value pairs;
determining, using the processing equipment, one or more metrics, wherein the one or more metrics are indicative of how the plurality of associated value pairs, when considered in two-dimensional space, are distributed about a curve; and
determining, using the processing equipment, a signal quality metric based on the one or more metrics.

8. The method of claim 7, wherein the curve comprises a line.

9. The method of claim 7, wherein determining the one or metrics comprises determining at least one of a standard error and a residual between the plurality of associated value pairs and a relationship represented by the curve.

10. The method of claim 7, wherein:
the first plurality of sample values and the second plurality of sample values comprise the same number of samples values; and
a time interval corresponding to the number of sample values is substantially equal to one half of a characteristic period of the photoplethysmograph signal, or an integer multiple thereof.

11. The method of claim 7, further comprising:
specifying a third segment of the photoplethysmograph signal based on a second time delay relative to the first segment, wherein the third segment comprises a third plurality of sample values; and
associating each of the plurality of associated value pairs with a correspond sample value of the third segment to generate a plurality of associated value triples, wherein the one or more metrics are indicative of how the plurality of associated value triples, when considered in three-dimensional space, are distributed about a plane.

12. A system for determining signal quality of a physiological signal, the system comprising:
processing equipment configured to:
receive a photoplethysmograph signal;
specify a first segment of the photoplethysmograph signal, wherein the first segment comprises a first plurality of sample values;
specify a second segment of the photoplethysmograph signal based on a time delay relative to the first segment, wherein the second segment comprises a second plurality of sample values;
associate each sample value of the first segment with a corresponding sample value of the second segment to generate a plurality of associated value pairs;
identify a subset of associated value pairs of the plurality of associated value pairs, wherein when the plurality of associated value pairs are considered in two-dimensional space, the subset of associated value pairs substantially correspond to a curve; and
determine a signal quality metric based on the subset of associated value pairs.

13. The system of claim 12, wherein the curve comprises a line.

14. The system of claim 12, wherein the processing equipment is further configured to:
identify at least one set of two associated values pairs that when considered in the two-dimensional space, lie on either side of the curve;
for each identified set of two associated value pairs, determine a respective intersection value by interpolating a point on the curve between the two associated value pairs; and
determine the signal quality metric further based on the at least one intersection value.

15. The system of claim 12, wherein the processing equipment is further configured to determine a variability metric associated with the subset of associated value pairs, wherein the variability metric is selected from the group comprising an average magnitude, a standard deviation, a median deviation, and an entropy value.

16. The system of claim 12, wherein the first plurality of sample values and the second plurality of sample values comprise the same number of samples values, and wherein a time interval corresponding to the number of sample values is greater than or substantially equal to a characteristic period of the photoplethysmograph signal.

17. The system of claim 12, wherein the processing equipment is further configured to:
specify a third segment of the photoplethysmograph signal based on a second time delay relative to the first segment, wherein the third segment comprises a third plurality of sample values; and
associate each of the plurality of associated value pairs with a correspond sample value of the third segment to generate a plurality of associated value triples;
identify a subset of associated value triples of the plurality of associated value triples, wherein when the plurality of associated value triples are considered in three-dimensional space, the subset of associated value triples substantially correspond to a surface in the three-dimensional space; and
determine the signal quality metric based on the subset of associated value triples.

18. A system for determining signal quality of a physiological signal, the system comprising:
processing equipment configured to:
receive a photoplethysmograph signal;
specify a first segment of the photoplethysmograph signal, wherein the first segment comprises a first plurality of sample values;
specify a second segment of the photoplethysmograph signal based on a time delay relative to the first segment, wherein the second segment comprises a second plurality of sample values;
associate each sample value of the first segment with a corresponding sample value of the second segment to generate a plurality of associated value pairs;
determine one or more metrics, wherein the one or more metrics are indicative of how the plurality of associated value pairs, when considered in two-dimensional space, are distributed about a curve; and
determine a signal quality metric based on the one or more metrics.

19. The system of claim 18, wherein the curve comprises a line.

20. The system of claim 18, wherein the processing equipment is further configured to determine at least one of a standard error and a residual between the plurality of associated value pairs and a relationship represented by the curve.

21. The system of claim 18, wherein:
the first plurality of sample values and the second plurality of sample values comprise the same number of samples values; and
a time interval corresponding to the number of sample values is substantially equal to one half of a characteristic period of the photoplethysmograph signal, or an integer multiple thereof.

22. The system of claim 18, wherein the processing equipment is further configured to:
specify a third segment of the photoplethysmograph signal based on a second time delay relative to the first segment, wherein the third segment comprises a third plurality of sample values; and
associate each of the plurality of associated value pairs with a correspond sample value of the third segment to generate a plurality of associated value triples, wherein the one or more metrics are indicative of how the plurality of associated value triples, when considered in three-dimensional space, are distributed about a plane.
